(19) United States
(12) Patent Application Publication
(10) Pub. No.: US 2010/0279944 A1
(43) Pub. Date: Nov. 4, 2010

(54) SUPPRESSION AND TREATMENT OF NEUROPATHIC PAIN

(76) Inventors: Ronald Mathison, Calgary (CA); Lynne C Weaver, London (CA)

Correspondence Address: Kenneth;Sharples, LAW OFFICE OF KENNETH K. SHARPLES, SENA PLAZA BUILDING, SUITE 54, 125 EAST PALACE AVE, SANTA FE, NM, 87501, US

(21) Appl. No.: 12/293,270

(22) PCT Filed: Mar. 29, 2007
(86) PCT No.: PCT/CA2007/000502

§ 371 Date: May 17, 2010

Related U.S. Application Data
(60) Provisional application No. 60/787,194, filed on Mar. 30, 2006.

(51) Int. Cl. A61K 38/08; A61K 38/05; A61P 25/00; A61P 3/10; A61P 35/00; A61P 25/28; A61P 9/10; A61K 38/06; A61K 38/07

(52) U.S. Cl. 514/18.2

(57) ABSTRACT

The invention provides a method for suppressing the development of or treating neuropathic pain in a subject comprising administering to the subject an effective amount of a peptide of the formula: X¹—X²—X₃ (I) or X¹—X² (II) wherein X¹ is an aromatic amino acid residue or is selected from the group consisting of 2-amino-hexanoic acid, 2-amino-heptanoic acid; 2-amino-octanoic acid; cyclohexyl-substituted 2-amino-ethanoic acid, cyclohexyl-substituted 2-amino-propanoic acid or 2-amino-butanoic acid and methionine; X² is an acidic amino acid; and in Formula (I), X³ is 1 to 3 amino acid residues which are the same or different and are aliphatic amino acid residues and the C-terminal amino acid is optionally amidated. These peptides may be used to treat a nerve injury or a spinal cord injury or to improve chronic neurological outcome after such injuries.

### FIELD OF THE INVENTION

The present invention relates to treatment of neuropathic pain and to the suppression of its development.

### BACKGROUND OF THE INVENTION

Pain is usually the natural consequence of tissue injury and in most cases resolves with the healing process. Two basic types of pain can be distinguished—acute and chronic. Acute or nociceptive pain is generally self-limiting and serves a protective biological function by warning of on-going tissue damage caused by noxious chemical, thermal and mechanical stimuli. The primary mediators of acute pain are algogenic substances (eg. histamine, bradykinin, substance P, etc.) stimulating receptors on A-delta and C-fibers which are located in skin, bone, connective tissue, muscle and viscera. Examples of nociceptive pain include: post-operative pain, pain associated with trauma, and the pain associated with arthritis.

Chronic pain, on the other hand, serves no protective biological function, and reflects either poor resolution of the painful stimuli, or is itself a disease process. Chronic pain is unrelenting and not self-limiting and can persist for years and even decades after the initial injury. Chronic pain is predominantly neuropathic in nature and involves damage either to the peripheral or central nervous systems. Neuropathic pain is caused by a primary lesion, malfunction or dysfunction in the nervous system. Such pain is described as “burning”, “electric”, “tingling”, and “shooting” in nature. The pain can be continuous or paroxysmal in presentation.

Neuropathic pain develops consequent to damage to or pathological changes in the peripheral or central nervous systems. Many nerve injuries arise from external causes such as the stresses and strains associated with sport and recreational activities, or they develop as a consequence of falls, sudden impacts or collisions occurring with motor vehicle crashes, assaults, or penetrating injuries with firearms or knives. Other nerve injuries have internal causes, resulting from strokes, viral infections, tumours, anoxia, hypoxia, toxins, degenerative diseases, allergies, stress, rheumatoid arthritis, fluid retention during pregnancy, menopause, or heart and kidney disease.

Early recognition and aggressive management of neuropathic pain is critical to successful outcome.

It is therefore desirable to initiate treatment with agents that can suppress the conditions that can eventually lead to the development of neuropathic pain. Selective inhibitors/antagonists of proinflammatory cytokines (tumor necrosis factor, interleukin-1, and interleukin-6), reactive oxygen species and complement have been shown to reduce allodynias caused by sciatic nerve inflammatory neuropathy (Twining C M, Sloane E M, Milligan E D, Chacur M, Martin D, Poole S, Marsh H, Maier S F, Watkins L R. Pain. 2004 July; 110 (1-2):299-309). Some agents, such as methyprednisolone sodium succinate which is used in some centres to treat traumatic nerve injury including spinal cord injury (SCI), is only modestly beneficial and may negate the beneficial actions of other therapies (Gorio A, Madaschi L, Di Stefano B, Carelli S, Di Giulio A M, De Biasi S, Coleman T, Cerami A, Brines M. Proc Natl Acad Sci USA. 2005 Nov. 8; 102 (45):16379-84). This result is surprising given that methyprednisolone decreases oxidative injury in a rat model of SCI (Kalayci M, Coskun O, Cagavi F, Kanter M, Armutcu F, Gul S, Acikgoz B. Neurochem Res. 2005 March; 30 (3):403-10), and indicates that some agents that suppress ROS production do not necessarily result in improved neuropathic outcomes. Also, leukotriene receptor antagonists that are used as anti-inflammatories and can suppress nociceptive pain are ineffective when used to treat chronic pain (Cartmell M T, O'Reilly D A, Porter C, Kingsnorth A N. J Hepatobiliary Pancreat Surg. 2004; 11(4):255-9).

Most neuropathic pain responds poorly to NSAIDS and opioid analgesics, although high doses of opioids may be effective in some patients. The much discussed selective serotonin reuptake inhibitors have not proven to be effective against neuropathic pain. Treatment by local anaesthetic block has only short-lived effects.

There remains a need for new treatments for neuropathic pain.

Submandibular glands secrete endocrine factors that are involved in the regulation of oral and systemic immune and inflammatory responses (Mathison et al., (1994), Immunol. Today, v. 15, pp. 527-532). One peptide that participates in homeostatic regulation is submandibular gland peptide T (SGP-T; amino acid sequence TDIFEGG), which shows biological effects at doses as low as 1 μg/kg (Mathison et al., (1997), Dig. Dis. Sci., v. 42, pp. 2378-2383). Investigation of structure activity relationships has shown that biological activity is maintained when SGP-T is truncated to the tripeptide FEG and many analogues and variants of FEG have also been shown to have biological activity (U.S. Pat. No. 6,852,697 and U.S. Pat. No. 6,586,403). These peptides have not previously been shown to have efficacy in the treatment or suppression of neuropathic pain.

### SUMMARY OF THE INVENTION

The present invention provides new methods for providing an improved neurological outcome in conditions or situations associated with the development of chronic or neuropathic pain or for treating chronic or neuropathic pain.

In one embodiment, the present invention provides a method for suppressing the development of or treating neuropathic pain in a subject, comprising administering to the subject an effective amount of a peptide of the formula:

X¹—X²—X³ (I)

or

X¹—X² (II)

wherein X¹ is an aromatic amino acid residue or is selected from the group consisting of 2-amino-hexanoic acid, 2-amino-heptanoic acid; 2-amino-octanoic acid; cyclohexyl-substituted 2-amino-ethanoic acid, cyclohexyl-substituted 2-amino-propanoic acid or 2-amino-butanoic acid and methionine;

X² is an acidic amino acid; and in Formula I, X³ is 1 to 3 amino acid residues which are the same or different and are aliphatic amino acid residues and the C-terminal amino acid is optionally amidated.

In another embodiment, the present invention provides a method for treating a nerve injury or a spinal cord injury comprising administering to the subject an effective amount of a peptide of the formula:

X¹—X²—X³ (I)

or

X¹—X² (II)

wherein X¹ is an aromatic amino acid residue or is selected from the group consisting of 2-amino-hexanoic acid, 2-amino-heptanoic acid; 2-amino-octanoic acid; cyclohexyl-substituted 2-amino-ethanoic acid, cyclohexyl-substituted 2-amino-propanoic acid or 2-amino-butanoic acid and methionine;

X² is an acidic amino acid; and in Formula I, X³ is 1 to 3 amino acid residues which are the same or different and are aliphatic amino acid residues and the C-terminal amino acid is optionally amidated.

In a further embodiment, the present invention provides a method for treating a subject suffering from a condition associated with the development of neuropathic pain comprising administering to the subject an effective amount of a peptide of the formula:

X¹—X²—X³ (I)

or

X¹—X² (II)

wherein X¹ is an aromatic amino acid residue or is selected from the group consisting of 2-amino-hexanoic acid, 2-amino-heptanoic acid; 2-amino-octanoic acid; cyclohexyl-substituted 2-amino-ethanoic acid, cyclohexyl-substituted 2-amino-propanoic acid or 2-amino-butanoic acid and methionine;

X² is an acidic amino acid; and in Formula I, X³ is 1 to 3 amino acid residues which are the same or different and are aliphatic amino acid residues and the C-terminal amino acid is optionally amidated.

In a further embodiment, the present invention provides a method for improving chronic neurological outcome after nerve injury or spinal cord injury in a subject, comprising administering to the subject an effective amount of a peptide of the formula:

X¹—X²—X³ (I)

or

X¹—X² (II)

wherein X¹ is an aromatic amino acid residue or is selected from the group consisting of 2-amino-hexanoic acid, 2-amino-heptanoic acid; 2-amino-octanoic acid; cyclohexyl-substituted 2-amino-ethanoic acid, cyclohexyl-substituted 2-amino-propanoic acid or 2-amino-butanoic acid and methionine;

X² is an acidic amino acid; and in Formula I, X³ is 1 to 3 amino acid residues which are the same or different and are aliphatic amino acid residues and the C-terminal amino acid is optionally amidated.

In further embodiments, X¹ is selected from the group consisting of phenylalanine, tyrosine, tryptophan, phenylglycine, nor-methylphenylalanine, cyclohexylalanine and norleucine.

In further embodiments, X² is glutamic acid.

In further embodiments, X³ is an amino acid residue selected from the group consisting of D or L-alanine, beta-alanine, valine, leucine, isoleucine, sarcosine, methionine, and gamma-amino butyric acid or is 1 to 3 glycine residues.

In a further embodiment, the peptide administered in the methods of the invention is at least one of L-Phenylalanine-L-Glutamic acid-Glycine, D-phenylalanine-D-glutamic acid-Glycine, L-Phenylalanine-L-Glutamic acid-L-Alanine, D-phenylalanine-D-glutamic acid-D-alanine, D-tyrosine-D-glutamic acid-Glycine, L-Phenylglycine-L-Glutamic acid-Glycine, L-NorMethylPhenylalanine-L-Glutamic acid-Glycine, L-Cyclohexylalanine-L-Glutamic acid-Glycine, D-cyclohexylalanine-D-glutamic acid-Glycine, L-Norleucine-L-Glutamic acid-Glycine, L-Methionine-L-Glutamic acid-Glycine, L-Phenylalanine-L-Glutamic acid-L-Methionine, L-Phenylalanine-L-Glutamic acid-L-Isoleucine, L-Phenylalanine-L-Glutamic acid-beta-Alanine, L-Phenylalanine-L-Glutamic acid-L-Sarcosine, L-Phenylalanine-L-Glutamic acid-Gamma-amino-butyric acid, L-Phenylalanine-L-Glutamic acid, D-phenylalanine-D-glutamic acid, D-tyrosine-D-glutamic acid, L-Cyclohexylalanine-L-Glutamic acid or D-cyclohexylalanine-D-glutamic acid.

In further embodiments, the peptide administered is L-Phenylalanine-L-Glutamic acid-Glycine, D-phenylalanine-D-glutamic acid-Glycine, L-Cyclohexyalanine-L-Glutamic acid-Glycine or D-cyclohexylalanine-D-glutamic acid-Glycine.

In further embodiments, the invention provides use of a peptide of the formula:

X¹—X²—X³ (I)

or

X¹—X² (II)

wherein X¹ is an aromatic amino acid residue or is selected from the group consisting of 2-amino-hexanoic acid, 2-amino-heptanoic acid; 2-amino-octanoic acid; cyclohexyl-substituted 2-amino-ethanoic acid, cyclohexyl-substituted 2-amino-propanoic acid or 2-amino-butanoic acid and methionine;

X² is an acidic amino acid; and in Formula I, X³ is 1 to 3 amino acid residues which are the same or different and are aliphatic amino acid residues, and the C-terminal amino acid is optionally amidated, for the preparation of a medicament for suppressing the development of or treating neuropathic pain.

In a further embodiment, the invention provides use of a peptide of the formula:

X¹—X²—X³ (I)

or

X¹—X² (II)

wherein X¹ is an aromatic amino acid residue or is selected from the group consisting of 2-amino-hexanoic acid, 2-amino-heptanoic acid; 2-amino-octanoic acid; cyclohexyl-substituted 2-amino-ethanoic acid, cyclohexyl-substituted 2-amino-propanoic acid or 2-amino-butanoic acid and methionine;

X² is an acidic amino acid; and in Formula I, X³ is 1 to 3 amino acid residues which are the same or different and are aliphatic amino acid residues, and the C-terminal amino acid is optionally amidated, for the preparation of a medicament for treating a nerve injury or a spinal cord injury.

In a further embodiment, the invention provides use of a peptide of the formula:

X¹—X²—X³ (I)

or

X¹—X² (II)

wherein X¹ is an aromatic amino acid residue or is selected from the group consisting of 2-amino-hexanoic acid, 2-amino-heptanoic acid; 2-amino-octanoic acid; cyclohexyl-substituted 2-amino-ethanoic acid, cyclohexyl-substituted 2-amino-propanoic acid or 2-amino-butanoic acid and methionine;

X² is an acidic amino acid; and in Formula I, X³ is 1 to 3 amino acid residues which are the same or different and are aliphatic amino acid residues, and the C-terminal amino acid is optionally amidated, for the preparation of a medicament for treating a subject suffering from a condition associated with the development of neuropathic pain

In a further embodiment, the invention provides use of a peptide of the formula:

X¹—X²—X³ (I)

or

X¹—X² (II)

wherein X¹ is an aromatic amino acid residue or is selected from the group consisting of 2-amino-hexanoic acid, 2-amino-heptanoic acid; 2-amino-octanoic acid; cyclohexyl-substituted 2-amino-ethanoic acid, cyclohexyl-substituted 2-amino-propanoic acid or 2-amino-butanoic acid and methionine;

X² is an acidic amino acid; and in Formula I, X³ is 1 to 3 amino acid residues which are the same or different and are aliphatic amino acid residues, and the C-terminal amino acid is optionally amidated, for the preparation of a medicament for improving chronic neurological outcome after nerve injury or spinal cord injury.

### SUMMARY OF THE DRAWINGS

Certain embodiments of the invention are described, reference being made to the accompanying drawings, wherein:

FIG. 1a shows the locomotor scores (Y-axis) of rats treated with the peptide feG (solid squares) and controls (open squares) at the indicated weeks post-spinal cord injury (X-axis). *p<0.05 compared with controls.

FIG. 1b shows the avoidance responses (Y-axis) of rats treated with feG (solid squares) and controls (open squares) at the indicated weeks post-spinal cord injury (X-axis). *p<0.05 compared with controls.

### DETAILED DESCRIPTION OF THE INVENTION

“Neuropathic pain”, as used herein, includes neuropathic pain, neurogenic pain (a term which is sometimes used in the literature as another name for neuropathic pain and sometimes to refer to transitory pain, the term “neuropathic” being reserved for more chronic conditions), allodynia, which is a disorder in which normally non-painful stimuli cause pain in affected subjects, hyperalgesia, in which normally painful stimuli cause a greater than normal level of pain in affected subjects, and phantom pain.

The tripeptide feG (D-phenylalanine-D-glutamic acid-Glycine) has been shown to improve chronic neurological outcomes after spinal cord injury in a rat model. A wide range of pharmacological interventions have been shown to have only short-lived effects on mechanical allodynia in spinal cord injured rats. In contrast, feG treatment had long-lasting effects that reduced mechanical allodynia induced below the injury site by as much as 50%. Motor function improved significantly after feG treatment and the scores of the treated group were still increasing when observations ended at seven weeks post injury, whereas the scores of control rats had reached a plateau at about three weeks after injury.

The peptide feG is representative of a group of peptides which share other biological activities of feG, as discussed above, in the background section. Such peptides include L-Phenylalanine-L-Glutamic acid-Glycine (FEG), D-phenylalanine-D-glutamic acid-Glycine (feG), L-Phenylalanine-L-Glutamic acid-L-Alanine (FEA), D-phenylalanine-D-glutamic acid-D-alanine (fea), D-tyrosine-D-glutamic acid-Glycine (yeG), L-Phenylglycine-L-Glutamic acid-Glycine ((Phg) EG), L-NorMethylPhenylalanine-L-Glutamic acid-Glycine ((NMeF) EG), L-Cyclohexylalanine-L-Glutamic acid-Glycine ((Cha) EG), D-cyclohexylalanine-D-glutamic acid-Glycine ((cha)eG), L-Norleucine-L-Glutamic acid-Glycine ((Nle)EG), L-Methionine-L-Glutamic acid-Glycine (MEG), L-Phenylalanine-L-Glutamic acid-L-Methionine (FEM), L-Phenylalanine-L-Glutamic acid-L-Isoleucine (FED, L-Phenylalanine-L-Glutamic acid-beta-Alanine (FE-β-alanine), L-Phenylalanine-L-Glutamic acid-L-Sarcosine (FE-Sarcosine), L-Phenylalanine-L-Glutamic acid-Gamma-amino-butyric acid (FE-Gamma-amino butyric acid), L-Phenylalanine-L-Glutamic acid (FE), D-phenylalanine-D-glutamic acid (fe), D-tyrosine-D-glutamic acid(ye), L-Cyclohexylalanine-L-Glutamic acid ((Cha) E) and D-cyclohexylalanine-D-glutamic acid ((cha)e).

The peptides used in the methods and uses of the invention may optionally be amidated at the C-terminal carboxyl group.

The peptide fdG (D-phenylalanine-D-aspartic acid-Glycine) was not active in improving chronic neurological outcomes as described herein. This suggests that the acidic amino acid of position X² of Formula I requires at least two methylene groups between the carboxyl group and the amino acid backbone, as in glutamic acid, for activity.

As used herein in relation to the structure of Formula I and Formula II, “acidic amino acid” means an acidic amino acid having two or more methylene groups between the carboxyl group and the amino acid backbone.

Suppressing the development of neuropathic pain means reducing the level of neuropathic pain which would otherwise develop in a subject who has suffered an injury or condition associated with subsequent development of neuropathic pain. Such injuries and conditions include spinal cord injuries, and peripheral nerve injuries arising from extreme stretching of a nerve, for example caused by joint dislocation, from decreased blood supply to a nerve, for example due to external pressure or from burning or cutting of the nerve due to trauma. Reducing the level of pain which would otherwise develop extends from partial reduction to complete reduction.

Treating neuropathic pain means ameliorating the symptoms of neuropathic pain in a subject suffering from neuropathic pain.

Nerve injury and neuropathic pain can arise as a result of a disease and such diseases include stroke, infection, tumours, anoxia, hypoxia, diabetes, metabolic syndrome, toxin exposure, degenerative diseases and allergic reactions.

An “effective amount” means an amount sufficient to produce amelioration of one or more symptoms of neuropathic pain or allodynia.

The subjects may be humans or non-human animals, including dogs, cats, horses, cows, sheep, rabbits, rats and mice and other domestic pets or farm animals.

Peptides for use in the methods of the invention may be prepared by any suitable peptide synthetic method, as known to those skilled in the art.

Chemical synthesis may be employed. For example, standard solid phase peptide synthetic techniques may be used. In standard solid phase peptide synthesis, peptides of varying length can be prepared using commercially available equipment. This equipment can be obtained, for example, from Applied Biosystems (Foster City, Calif.). The reaction conditions in peptide synthesis are optimized to prevent isomerization of stereochemical centres, to prevent side reactions and to obtain high yields. The peptides are synthesized using standard automated protocols, using t-butoxycarbonyl-alpha-amino acids, and following the manufacture's instructions for blocking interfering groups, protecting the amino acid to be reacted, coupling, deprotecting and capping of unreacted residues. The solid support is generally based on a polystyrene resin, the resin acting both as a support for the growing peptide chain, and as a protective group for the carboxy terminus. Cleavage from the resin yields the free carboxylic acid. Peptides are purified by HPLC techniques, for example on a preparative C18 reverse phase column, using acetonitrile gradients in 0.1% trifluoroacetic acid, followed by vacuum drying. The required peptides can also be produced by liquid phase peptide chemistry.

Peptides may also be produced by recombinant synthesis. A DNA sequence encoding the desired peptide is prepared and subcloned into an expression plasmid DNA. Suitable mammalian expression plasmids include pRC/CMV from Invitrogen Inc. The gene construct is expressed in a suitable cell line, such as a Cos or CHO cell line and the expressed peptide is extracted and purified by conventional methods. Suitable methods for recombinant synthesis of peptides are described in Sambrook et al., (1989), “Molecular Cloning” Cold Spring Harbor, Lab. Press, Cold Spring Harbor, N.Y. Derivatives of a peptide may be prepared by similar synthetic methods. Examples of side chain modifications contemplated by the present invention include modification of amino groups such as by reductive alkylation by reaction with an aldehyde followed by reduction with NaBH₄; amidation with methylacetimidate; acetylation with acetic anhydride; carbamylation of amino groups with 2, 4, 6, trinitrobenzene sulfonic acid (TNBS); alkylation of amino groups with succinic anhydride and tetrahydrophthalic anhydride; and pyridoxylation of lysine with pyridoxal-5′-phosphate followed by reduction with NaBH₄.

In a number of situations, the likely development of neuropathic pain can be anticipated, as a result of a particular event such as spinal cord injury, injuries arising from accidents, motor vehicle collisions, assaults and recreational activities, strokes or ingestion of toxins. Subjects who have experienced such an event and are at risk of developing neuropathic pain are candidates for treatment by the methods of the invention, initiated as soon as possible after the event, to suppress or reduce the development of neuropathic pain. Treatment could be continued daily for any desired period of time, for example, from several days to several weeks.

Peptides may be administered therapeutically by injection or by oral, nasal, buccal, sub-lingual, rectal, vaginal, transdermal or ocular routes in a variety of formulations, as is known to those of skill in the art.

For oral administration, various techniques can be used to improve stability, based for example on chemical modification, formulation and use of protease inhibitors. Stability can be improved if synthetic amino acids are used, such as betidamino acids, or if metabolically stable analogues are prepared.

Formulation may be, for example, in water/oil emulsion or in liposomes for improved stability. Orally administered peptides may be accompanied by protease inhibitors such as aprotinin, soybean trypsin inhibitor or FK-448, to provide protection for the peptide. Suitable methods for preparation of oral formulations of peptide drugs can be found, for example, in Lundin et al., (1986), Life Sci., v. 38, pp. 703-709; Saffran et al., (1979), Can J. Biochem., v. 57, pp. 548-553; and Vilhardt et al., (1986), Gen Pharmacol., v. 17, pp. 481-483.

Due to its high surface area and extensive vascular network, the nasal cavity provides a good site for absorption of both lipophilic and hydrophilic drugs, especially when coadministered with absorption enhancers. The nasal absorption of peptide-based drugs can be improved by using aminoboronic acid derivatives, amastatin, and other enzyme inhibitors as absorption enhancers and by using surfactants such as sodium glycolate, as described Amidon et al., (1994), Rev. Pharmacol. Toxicol., v. 34, pp. 321-341. The transdermal route provides good control of delivery and maintenance of the therapeutic level of drug over a prolonged period of time (Amidon et al., supra; Choi et al., (1990), Pharm. Res., v. 7, 1099-1106). A means of increasing skin permeability is desirable, to provide for systemic access of peptides. For example, iontophoresis can be used as an active driving force for charged peptides or chemical enhancers such as the nonionic surfactant n-decylmethyl sulfoxide (NDMS) can be used.

Peptides may also be conjugated with water soluble polymers such as polyethlene glycol, dextran or albumin or incorporated into drug delivery systems such as polymeric matrices to increase plasma half-life.

More generally, formulations suitable for particular modes of administration of peptides are described, for example, in “Peptide and Protein Drug Delivery”, (1991), Lee, V. H. L., Marcel Dekker, Inc., N.Y., N.Y. or in “Protein Formulation and Delivery (Drugs and the Pharmaceutical Sciences: a Series of Text books and Monographs”, (2000) McNally, E. J., Marcel Dekker, Inc., N.Y., N.Y.)

The particular dosage required in a given subject can be determined by the attending physician. A starting dosage in the range of 1 μg-1000 μg peptide/kg body weight can be employed, with adjustment of the dosage based on the response of a particular subject, as understood by those of ordinary skill in the art.

The peptides may also be formulated as food supplements by their addition to food products or beverage products. The use of peptides as food additives and their incorporation into food or beverage products is well known to those of skill in the food processing art. Where the peptides contain only natural amino acids, these products are attractive to those who favour natural medicines and natural health products.

### EXAMPLES

The examples are described for the purposes of illustration and are not intended to limit the scope of the invention.

Methods of chemistry, biochemistry and immunology referred to but not explicitly described in this disclosure and examples are reported in the scientific literature and are well known to those skilled in the art.

### Methods

### Animal Preparation

Seventeen individually housed male Wistar rats (Charles River, St. Constant, Quebec, 250-320 g) were used to assess mechanical allodynia and hind-limb locomotion. Males were used in these studies to avoid the confounding variable of hormonal cycles on the assessments of neuropathic pain. All protocols were carried out in accordance with the guidelines set forth by the Canadian Guide to Care and Use of Experimental Animals. All experiments conformed to international guidelines on the ethical use of animals and minimized the number of animals used and their suffering. All rats received pre-operative medication and halothane anesthesia for surgical interventions to accomplish clip compression spinal cord injury (SCI), as described previously (Gris et al., (2004), J. Neurosci., v. 24, pp. 4043-4051; Weaver et al., (2001), J. Neurotrauma, v. 18, pp. 1107-1119). A dorsal laminectomy was performed to expose the 12th thoracic (T12) spinal cord segment. The cord was injured by clip compression for 60 sec without disrupting the dura or adjacent dorsal roots. A modified aneurysm clip (Toronto Western Research Institute, University of Toronto, Toronto, Ontario, Canada) calibrated at 35 g was used at the T12 segment to produce a moderate injury. The rats received postoperative care as described previously (Gris, supra). This clip compression model is accepted in the art as a model of spinal cord injury which mimics key patho-physiological features commonly seen in human spinal cord injury. The treated rats were studied for seven weeks to assess motor function and mechanical allodynia as previously described (Bruce et al., Exp. Neurol., v. 178, pp. 33-48; Gris supra).

Protocol for Treatment with feG

Animals were blindly assigned to a control or treatment group. The treatment group (n=7) received the feG [phenylalanine-(D) glutamate-(D) Glycine] peptide (200 μg/kg). Control injured rats received either normal saline (n=4) or phenylalanine-(D) aspartate-(D) Glycine (fdG), an inactive peptide (n=6; 200 μg/kg). The saline-treated rats had been part of a pilot study for this project and, as the results obtained from these animals were not different from those of the fdG-treated rats, the two control groups were combined. The peptides were injected intravenously via the tail vein as six consecutive bolus doses at 2, 12, 24, 36, 48 and 60 h after SCI. Anaesthesia was not required for these injections. This study was completed at seven weeks after SCI. All testing and data analysis were carried out with the investigator blinded to the treatment received by each animal.

Animals treated with feG, fdG or saline followed a typical recovery from the T12 injury. No overt side effects of this treatment were noted. The rats began to eat and drink within 24 h of the injury, moved about in their cages using their forelimbs, and eventually with their hind limbs. The rats gained weight in the seven post-SCI weeks, the controls weighing 336±10.4 g and the feG-treated rats weighing 359±15.8 g at the termination of the study.

### Neurological Outcomes

Locomotor recovery of animals with injury at T12 was assessed using the 21 point Basso, Beattie, and Bresnahan (BBB) open-field locomotor score (Basso et al., (1995), J. Neurotrauma, v. 12, pp. 1-21) from seven days to seven weeks after SCI. Scores for left and right hind limbs were averaged. These scores were recorded twice per week and the average for each week calculated.

During the week prior to the 112 SCI, rats were tested for mechanical allodynia on the plantar surface of the hindpaws. They were then tested again during the third to seventh weeks after SCI as described previously (Oatway et al., (2005), J. Neurosci., v. 25, pp. 637-647). Mechanical allodynia is neuropathic pain in which stimuli that are normally non-noxious generate avoidance responses. Using a modified Semmes Weinstein filament, calibrated to generate a force of 15 mN, rats were tested for avoidance responses by stimulating the plantar surface of the hindpaws once per week. Stimuli were applied 5 seconds apart and the number of avoidance responses to ten stimuli was recorded. Avoidance responses were defined as flinching, escape, paw withdrawal and/or licking, vocalization or abnormal aggressive behaviours and indicated that the rat perceived the stimulus as noxious.

### Statistical Analysis

All statistical analyses were performed using GB-Stat V7.0 software (Dynamic Microsystems Incorporated). BBB scores were analyzed using two-way analysis of variance (ANOVA) with repeated measures followed by the Fisher's LSD (protected t) test for multiple comparisons, and regression analysis followed by a one-way ANOVA for comparison of the homogeneity of slopes. Paw scores between groups were assessed by comparing the mean area under the time vs. response curve using a one-way ANOVA. Lesion analysis was conducted using a two-way ANOVA with repeated measures to assess treatment effects. Significant differences were accepted at P<0.05 and variability is expressed as a standard error of the mean.

### Example 1

feG Treatment after SCI Improves Locomotor Function

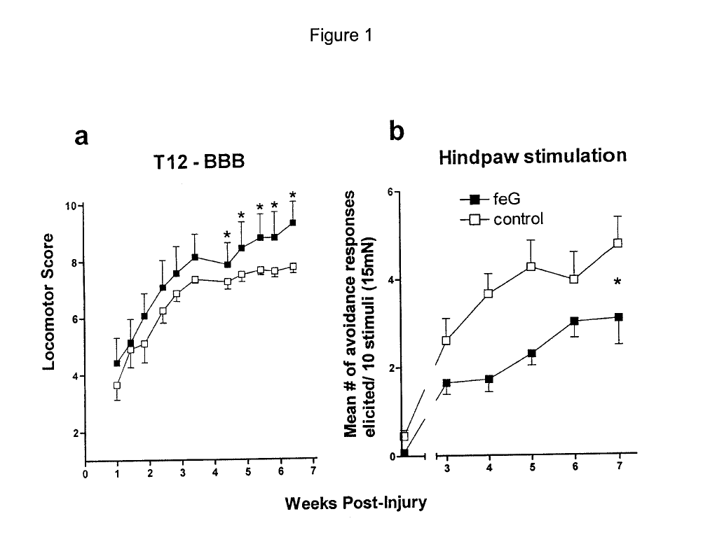

During the first four weeks of study, mean BBB scores of the feG-treated rats were not significantly greater than those of the control group using a two-way ANOVA with repeated measures [interaction F=0.23 (5.75), P=0.949). However from 31-45 days after SCI, the slope of the time vs. motor score recovery curve (0.21) decreased in control groups when compared to the respective slopes in the first 28 days (1.49). The decrease was significant when assessed by linear regression followed by comparison of the slopes [F=74.12 (1.9), P=0.00001]. Likewise the slope of the recovery curve in feG-treated rats (0.64) decreased compared to that of the first 28 days [1.57, F=23.82 (1.8) P=0.001]. Moreover, when assessed by linear regression, the slope of the line expressing time vs. motor score (from 31-45 days) was significantly greater in the feG-treated animals (0.64) than in the controls [0.21, F=16.21 (1.10), P=0.00241]. The control group had reached a plateau whereas the scores of the feG group kept increasing. During this interval, the mean BBB scores for the feG-treated rats were significantly greater than those of the controls using a two-way ANOVA with repeated measures [interaction F=2.65 (4.60), P=0.042]. Accordingly, treatment with the peptide feG caused a delayed but significant improvement in motor function after SCI. BBB scores of control rats had a maximum score 7.8±0.2 points, while scores of feG-treated rats reached a maximum score of 9.1±0.7 points at seven weeks after SCI (FIG. 1a). The difference between a score of eight and nine points is notable, because although both signify an intermediate phase of recovery, only a score of nine is awarded for weight support with hind-limbs.

### Example 2

### feG Treatment Reduces Mechanical Allodynia After SCI

A modified Semmes Weinstein filament calibrated to generate a force of 15 mN was used to test for avoidance responses, by probing the plantar surface of the hindpaws. Rats tested on the hindpaw before SCI rarely exhibited avoidance behavior (FIG. 1b). Paw testing resumed at three weeks after SCI, and all rats had a higher incidence of avoidance behavior in response to ten stimuli. This pattern of behavior is consistent with the development of mechanical allodynia. Treatment with the feG peptide decreased incidences of avoidance behavior in response to ten stimuli applied on the plantar surface of the hindpaws seven weeks after SCI (FIG. 1b). The mean area under the time vs. response curve for feG treated rats was significantly lower than the mean area under the curve for control rats [F=8.32 (1.15) P=0.011]. Control rats demonstrated 4.8±0.6 avoidance responses to ten stimuli by seven weeks after SCI whereas rats treated with feG had fewer, with only 3.1±0.6 avoidance responses at this time.

1. A method for suppressing the development of or treating neuropathic pain in a subject, comprising administering to the subject an effective amount of a peptide of the formula:
X¹—X²—X³ (I)
or
X¹—X² (II)
wherein X¹ is an aromatic amino acid residue or is selected from the group consisting of 2-amino-hexanoic acid, 2-amino-heptanoic acid; 2-amino-octanoic acid; cyclohexyl-substituted 2-amino-ethanoic acid, cyclohexyl-substituted 2-amino-propanoic acid or 2-amino-butanoic acid and methionine;
X² is an acidic amino acid; and in Formula I, X³ is 1 to 3 amino acid residues which are the same or different and are aliphatic amino acid residues and the C-terminal amino acid is optionally amidated.

2. A method for treating a nerve injury or a spinal cord injury comprising administering to the subject an effective amount of a peptide of the formula:
X¹—X²—X³ (I)
or
X¹—X² (II)
wherein X¹ is an aromatic amino acid residue or is selected from the group consisting of 2-amino-hexanoic acid, 2-amino-heptanoic acid; 2-amino-octanoic acid; cyclohexyl-substituted 2-amino-ethanoic acid, cyclohexyl-substituted 2-amino-propanoic acid or 2-amino-butanoic acid and methionine;
X² is an acidic amino acid; and in Formula I, X³ is 1 to 3 amino acid residues which are the same or different and are aliphatic amino acid residues and the C-terminal amino acid is optionally amidated.

3. A method for treating a subject suffering from a condition associated with the development of neuropathic pain comprising administering to the subject an effective amount of a peptide of the formula:
X¹—X²—X³ (I)
or
X¹—X² (II)
wherein X¹ is an aromatic amino acid residue or is selected from the group consisting of 2-amino-hexanoic acid, 2-amino-heptanoic acid; 2-amino-octanoic acid; cyclohexyl-substituted 2-amino-ethanoic acid, cyclohexyl-substituted 2-amino-propanoic acid or 2-amino-butanoic acid and methionine;
X² is an acidic amino acid; and in Formula I, X³ is 1 to 3 amino acid residues which are the same or different and are aliphatic amino acid residues and the C-terminal amino acid is optionally amidated.

4. The method of claim 3 wherein the condition is spinal cord injury.

5. The method of claim 3 wherein the condition is a nerve injury.

6. The method of claim 5 wherein the nerve injury resulted from an event selected from the group consisting of a sports injury, a fall, an accident and a wound.

7. The method of claim 5 wherein the nerve injury resulted from a disease.

8. The method of claim 7 wherein the disease is selected from the group consisting of stroke, infection, tumour, anoxia, hypoxia, diabetes, metabolic syndrome, toxin exposure, a degenerative disease, and an allergic reaction.

9. A method for improving chronic neurological outcome after nerve injury or spinal cord injury in a subject, comprising administering to the subject an effective amount of a peptide of the formula:
X¹—X²—X³ (I)
or
X¹—X² (II)
wherein X¹ is an aromatic amino acid residue or is selected from the group consisting of 2-amino-hexanoic acid, 2-amino-heptanoic acid; 2-amino-octanoic acid; cyclohexyl-substituted 2-amino-ethanoic acid, cyclohexyl-substituted 2-amino-propanoic acid or 2-amino-butanoic acid and methionine;
X² is an acidic amino acid; and in Formula I, X³ is 1 to 3 amino acid residues which are the same or different and are aliphatic amino acid residues and the C-terminal amino acid is optionally amidated.

10. The method of any one of claims 1 to 3 and 9 wherein X¹ is selected from the group consisting of phenylalanine, tyrosine, tryptophan, phenylglycine, nor-methylphenylalanine, cyclohexylalanine and norleucine.

11. The method of any one of claims 1 to 3 and 9 wherein X² is glutamic acid.

12. The method of any one of claims 1 to 3 and 9 wherein X³ is an amino acid residue selected from the group consisting of D or L-alanine, beta-alanine, valine, leucine, isoleucine, sarcosine, methionine, and gamma-amino butyric acid or is 1 to 3 glycine residues.

13. The method of any one of claims 1 to 3 and 9 wherein the administered peptide is selected from the group consisting of
L-Phenylalanine-L-Glutamic acid-Glycine,
D-phenylalanine-D-glutamic acid-Glycine,
L-Phenylalanine-L-Glutamic acid-L-Alanine,
D-phenylalanine-D-glutamic acid-D-alanine,
D-tyrosine-D-glutamic acid-Glycine,
L-Phenylglycine-L-Glutamic acid-Glycine,
L-NorMethylPhenylalanine-L-Glutamic acid-Glycine,
L-Cyclohexylalanine-L-Glutamic acid-Glycine,
D-cyclohexylalanine-D-glutamic acid-Glycine,
L-Norleucine-L-Glutamic acid-Glycine,
L-Methionine-L-Glutamic acid-Glycine
L-Phenylalanine-L-Glutamic acid-L-Methionine,
L-Phenylalanine-L-Glutamic acid-L-Isoleucine,
L-Phenylalanine-L-Glutamic acid-beta-Alanine,
L-Phenylalanine-L-Glutamic acid-L-Sarcosine,
L-Phenylalanine-L-Glutamic acid-Gamma-amino-butyric acid,
L-Phenylalanine-L-Glutamic acid,
D-phenylalanine-D-glutamic acid,
D-tyrosine-D-glutamic acid,
L-Cyclohexylalanine-L-Glutamic acid, and
D-cyclohexylalanine-D-glutamic acid.

14. The method of any one of claims 1 to 3 and 9 wherein the administered peptide is L-Phenylalanine-L-Glutamic acid-Glycine.

15. The method of any one of claims 1 to 3 and 9 wherein the administered peptide is D-phenylalanine-D-glutamic acid-Glycine.

16. The method of any one of claims 1 to 3 and 9 wherein the administered peptide is L-Cyclohexyalanine-L-Glutamic acid-Glycine.

17. The method of any one of claims 1 to 3 and 9 wherein the administered peptide is D-cyclohexylalanine-D-glutamic acid-Glycine.

18-29. (canceled)

30. The method of any one of claims 1 to 3 and 9 wherein the subject is a human subject.

31. The method of claim 10 wherein X² is glutamic acid.

32. The method of claim 10 wherein X³ is an amino acid residue selected from the group consisting of D or L-alanine, beta-alanine, valine, leucine, isoleucine, sarcosine, methionine, and gamma-amino butyric acid or is 1 to 3 glycine residues.

33. The method of claim 11 wherein X³ is an amino acid residue selected from the group consisting of D or L-alanine, beta-alanine, valine, leucine, isoleucine, sarcosine, methionine, and gamma-amino butyric acid or is 1 to 3 glycine residues.

34. The method of claim 31 wherein X³ is an amino acid residue selected from the group consisting of D or L-alanine, beta-alanine, valine, leucine, isoleucine, sarcosine, methionine, and gamma-amino butyric acid or is 1 to 3 glycine residues.
